(19) United States
(12) Patent Application Publication
(10) Pub. No.: US 2007/0128121 A1
(43) Pub. Date: Jun. 7, 2007

(54) Polymer hydrogels for controlled release and absorption of biocides

(75) Inventors: Crystal G. Densmore, Los Alamos, NM (US); Thomas W. Robison, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: The Regents of the University of California

(21) Appl. No.: 11/295,369

(22) Filed: Dec. 5, 2005

(51) Int. Cl. A61K 49/00; A61K 9/22

(52) U.S. Cl. 424/10.1; 424/468

(57) ABSTRACT

A material that is capable of releasing an active agent in a controlled manner. The material includes a hydrogel vehicle and an active agent dispersed throughout the hydrogel. When brought into contact with an aqueous release media, the active agent is released from the hydrogel vehicle into the release media. Characteristics of the hydrogel, including composition and surface area, control the release of the agent. Methods of forming the material, dispensing the active agent, and removing the active agent from an aqueous solution are also described.

### STATEMENT REGARDING FEDERAL RIGHTS

This invention was made with government support under Contract No. W-7405-ENG-36, awarded by the U.S. Department of Energy. The government has certain rights in the invention.

### BACKGROUND OF INVENTION

The invention relates to the dispensing of active agents, such as biocides, therapeutic agents, dye, surfactants, and the like. More particularly, the invention relates to a material that dispenses such agents. Even more particularly, the invention relates to a material that dispenses such agents in a controllable fashion.

There is an interest in dispensing certain reactive agents, such as biocides, therapeutic agents, dye, surfactants, and the like, in controllable doses and over extended periods of time. While many such agents are being examined, biocides such as, for example, cetylpyridinium chloride (CPC), are of particular interest due to their utility in diverse fields, including medicine, the food industry, and homeland security. Such biocides are typically dispensed from a variety of media. In most instances, either only a portion of the total amount of biocide is released or the release cannot be sustained over an extended period of time.

Presently no material is capable of releasing such active agents in a controlled manner or releasing high levels of active agent. Therefore, what is needed is a material that is capable of releasing an active agent in a controlled manner in either a single high level burst or over an extended period of time. What is also needed is a material that can accept and dispense large amounts of active agent.

### SUMMARY OF INVENTION

The present invention meets these and other needs by providing a material that is capable of releasing an active agent in a controlled manner. The material comprises a hydrogel vehicle and an active agent dispersed throughout the hydrogel. When brought into contact with an aqueous release media, the active agent is released. Characteristics of the hydrogel, including composition and surface area, control the release of the agent. Methods of forming the material, dispensing the active agent, and removing the active agent from an aqueous solution are also described.

Accordingly, one aspect of the invention is to provide a material for controlled release of an active agent. The material comprises: a vehicle comprising a hydrogel, wherein the hydrogel comprises a hydrophobic portion and a hydrophilic portion and is substantially free of organic solvents; and an active agent dispersed in the hydrogel, wherein the hydrogel releases the active agent in a controllable manner.

A second aspect of the invention is to provide a vehicle for release of an active agent. The vehicle comprises a hydrogel selected from the group consisting of hydroxyethyl methacrylate-based hydrogels and hydroxyl ethyl acrylate-based hydrogels, wherein the hydrogel comprises a hydrophobic portion and a hydrophilic portion, wherein the hydrogel regulates release of the active agent.

A third aspect of the invention is to provide a material for controlled release of an active biocide. The material comprises: a vehicle comprising a hydrogel selected from the group consisting of hydroxyethyl methacrylate-based hydrogels and hydroxyl ethyl acrylate-based hydrogels, wherein the hydrogel comprises a hydrophobic portion and a hydrophilic portion; and an active biocide dispersed in the hydrogel, wherein the hydrogel regulates release of the active biocide.

A fourth aspect of the invention is to provide a method of forming a material comprising a vehicle, the vehicle comprising a hydrogel selected from the group consisting of hydroxyethyl methacrylate-based hydrogels and hydroxyl ethyl acrylate-based hydrogels, and an active agent dispersed in the hydrogel. The method comprises the steps of: providing a monomer, a crosslinker, an initiator, and the active agent; forming an aqueous solution of the monomer, the crosslinker, the initiator, and the active agent; and forming the hydrogel by polymerizing the monomer in the solution, wherein the active agent is dispersed throughout the hydrogel.

A fifth aspect of the invention is to provide a method of dispensing an active agent. The method comprises the steps of: providing a vehicle, the vehicle comprising a hydrogel selected from the group consisting of hydroxyethyl methacrylate-based hydrogels and hydroxyl ethyl acrylate-based hydrogels, and the active agent dispersed in the hydrogel; and contacting the vehicle with an aqueous release medium; wherein the active agent is released from the vehicle into the release medium.

A sixth aspect of the invention is to provide a method of absorbing an active agent from an aqueous medium containing the active agent. The method comprises the steps of: providing a hydrogel selected from the group consisting of hydroxyethyl methacrylate-based hydrogels and hydroxyl ethyl acrylate-based hydrogels; and contacting the hydrogel with the aqueous medium, wherein the active agent is absorbed by the hydrogel from the aqueous medium.

These and other aspects, advantages, and salient features of the present invention will become apparent from the following detailed description, the accompanying drawings, and the appended claims.

### BRIEF DESCRIPTION OF THE DRAWINGS

FIG. 1 is a plot comparing cetylpyridinium chloride (CPC) release in deionized water with varying degrees of anionic monomer 2-acrylamido-2-methyl-1-propanesulfonic acid (AMPSA) present in the polymer hydrogel;

FIG. 2 is a plot comparing CPC release in pH 7.41 phosphate buffer with varying degrees of anionic monomer AMPSA present in the polymer hydrogel;

FIG. 3 is a plot comparing CPC release in deionized water with varying degrees of crosslinker diethyleneglycol dimethyacrylate (DEGDMA) in polymer hydrogel;

FIG. 4 is a plot of CPC release as a function of regeneration cycle;

FIG. 5 is a plot of CPC release in deionized water with varying hydrogel surface areas;

FIG. 6 is a plot comparing CPC release in deionized water with varying degrees of CPC loading;

FIG. 7 is a flow chart for a method of making a hydrogel capable of controlled release of an active agent;

FIG. 8 is a flow chart for a method of dispensing an active agent from a hydrogel in a controlled manner;

FIG. 9 is a flow chart for a method of absorbing an active agent from an aqueous medium;

FIG. 10 is a plot of CPC absorption in deionized water under static and dynamic conditions;

FIG. 11 is a plot of CPC absorption in pH 7.41 phosphate buffer under static and dynamic conditions; and

FIG. 12 is a plot of CPC release for different release cycles.

### DETAILED DESCRIPTION

In the following description, like reference characters designate like or corresponding parts throughout the several views shown in the figures. It is also understood that terms such as “top,” “bottom,” “outward,” “inward,” and the like are words of convenience and are not to be construed as limiting terms. In addition, whenever a group is described as either comprising or consisting of at least one of a group of elements and combinations thereof, it is understood that the group may comprise or consist of any number of those elements recited, either individually or in combination with each other.

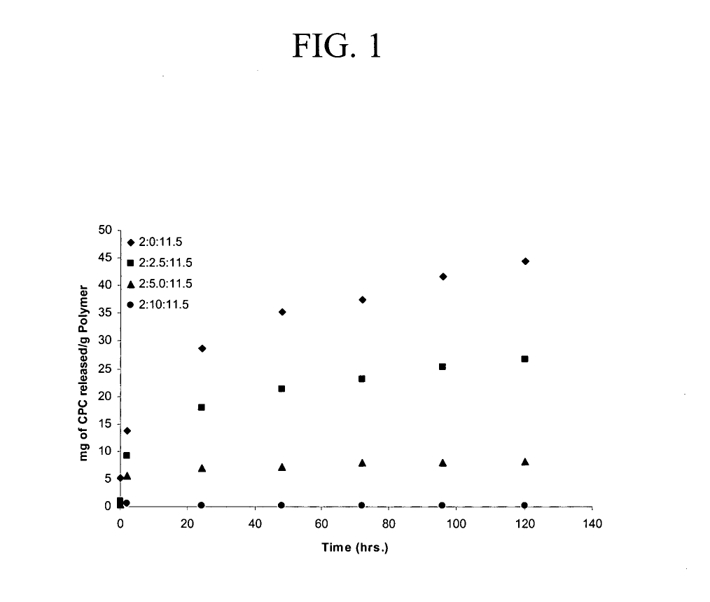

Referring to the drawings in general and to FIG. 1 in particular, it will be understood that the illustrations are for the purpose of describing a particular embodiment of the invention and are not intended to limit the invention thereto.

One aspect of the invention is to provide a material that is capable of releasing an active agent in a controllable manner such as, for example, in a single high dosage or “burst,” or over a prolonged period of time. Active agents that may be dispensed by such a material include, but are not limited to, dyes, biocides, therapeutic agents, and the like. The material comprises a vehicle comprising a hydrogel that is substantially free of organic solvents and at least one active agent dispersed throughout the hydrogel. When brought into contact with a release media, the material releases the active agent into the media.

In one embodiment, the hydrogel comprising the vehicle is a hydroxyethyl methacrylate (also referred to herein as “HEMA”) based hydrogel such as, for example, 2-hydroxyethyl methacrylate, having a hydrophilic portion and a hydrophobic portion. HEMA-based hydrogels are attractive for a variety of applications due to their high water content, non-toxicity, and biocompatibility. Alternatively, the hydrogel may be a hydroxyl ethyl acrylate-based hydrogel. The hydrophobic portion of the hydrogel is a non-toxic compound having the formula CH₂CHR₁CO₂R₂ where R₁, independently and at each occurrence, is one of —H, —CH₃, and —CH₂CH₃, and R₂, independently and at each occurrence, is one of an alkyl group, a cycloalkyl group, a hydroxyalkyl group, an aminoalkyl group, and an aryl group.

The hydrophilic portion of the hydrogel is a non-toxic compound and, in one embodiment, has the formula CH₂CHR₁CONR₂R₃, where R₁, independently and at each occurrence, is one of —H, —CH₃, and —CH₂CH₃, R₂, independently and at each occurrence, is one of —H and an alkyl group, and R₃, independently and at each occurrence, is one of —H and an alkyl group. Alternatively, the hydrophilic portion has the formula CH₂CH₂PO₃R₂, where R₂, independently and at each occurrence, is one of —H, —CH₃, and —CH₂CH₃. In another embodiment, the hydrophilic portion is CH₂CH₂SO₃H. In a fourth embodiment, the hydrophilic portion is CH₂CHR₁CO₂R₂ where R₁, independently and at each occurrence, is one of —H, —CH₃, and —CH₂CH₃, and R₂, independently and at each occurrence, is one of —H, —CH₃, and —CH₂CH₃.

The active agent may be a biocide or other cationic agent. In one embodiment, the biocide is an alkylpyridinium salt having the structure 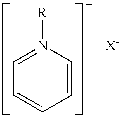
The alkylpyridinium salt may comprise an alkyl group having from 8 to 18 carbon atoms. The anion (X⁻) of the salt is capable of making the salt water-soluble. Such anions include, but are not limited to, chloride, bromide, and sulfate ions. In one embodiment, the alkylpyridinium salt is cetylpyridinium chloride (also referred to herein as “CPC”).

In another embodiment, the biocide is a quaternary aliphatic salt having the structure 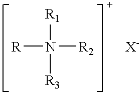
where R, independently and at each occurrence, is one of a benzyl group, a lower alkyl benzyl group (i.e., a short aliphatic chain (C₁-C₁₆) separates the benzyl group from N), a C₁-C₄ alkyl group, and a C₁-C₄ hydroxyalkyl group; R₁, independently and at each occurrence, is one of a C₁-C₄ alkyl group and a C₁-C₄ hydroxyalkyl group; R₂, independently and at each occurrence, is a C₈-C₂₄ alkyl group; and R₃, independently and at each occurrence, is one of a C₁-C₄ alkyl group, a C₁-C₄ hydroxyalkyl group, a C₈-C₂₄ alkyl group, and a C₈-C₂₄ hydroxyalkyl group. The anion X⁻ is an anion capable of imparting water solubility to the aliphatic quaternary ammonium salt. Non-limiting examples of biocidal aliphatic quaternary ammonium salts include (C₈-C₁₈) alkyl-dimethylbenzylammonium chloride and (C₈-C₁₈) alkyl-trimethylammonium bromide.

In another embodiment, the biocide is a phenolic compound such as, but not limited to, phenol, o-phenylphenol, 4-chloro-3,5-dimethylphenol, 2-chloro-3,5-dimethylphenol, m-cresol, o-cresol, p-cresol, and water-soluble salts thereof.

Other biocides include benzalkonium (C₈-C₁₈) chloride, triclocarban, triclosan, chlorhexidene, chlorhexidene gluconate, hexachlorophene, phenolics, dibromopropamidine isethionate, 2-ethoxyethanol, 1-phenylethanol, 1,3,5-trioxane, formaldehyde, paraformaldehyde, o-phthaldehyde, glutaraldehyde, and the like.

Other types of active agents that may be included in the material include dyes, surfactants, and therapeutic agents such as anti-inflammatory agents, respiratory agents, antibiotics, and the like. Examples of such dyes include alizarin, alizarin red S monohydrate, bromocresol green, bromocresol purple, bromophenol blue, bromothymol blue, congo red, cresol red, methyl orange, methyl red, and the like. Surfactants include, but are not limited to, biocides, such as those described hereinabove. Non-limiting examples of therapeutic agents include anti-inflammatory agent such as ibuprofen, respiratory agents such as theofylline, antibiotics such as cefazoline and sodium salt of cefazoline, and cytarabine, which is used in treatment of certain types of leukemia.

Release of the active agent from the vehicle takes place in a controlled fashion. In one embodiment, the active agent is released as an initial “burst:” a large release of active agent followed by a lower, stabilized release rate. For example, burst release for CPC is generally complete within 24 hours before a stable linear release is achieved. The amount of CPC released during the burst release period decreases with an increase in either the anionic monomer 2-acrylamido-2-methyl-1-propanesulfonic acid (also referred herein as “AMPSA”), or the crosslinker diethyleneglycol dimethyacrylate (also referred to herein as “DEGDMA”). The amount of CPC released during the burst period dramatically increases when the CPC loading is increased and when the CPC is loaded within an uncharged HEMA-based hydrogel.

One possible reason for burst release is dissolution of unbound material present on the surface of the hydrogel. In this case, the burst release may be reduced by surface extraction of the active agent. For example, the burst release of CPC from an uncharged HEMA-based hydrogel is largely attributed to CPC present on the surface of the hydrogel.

In another embodiment, active agent release is sustained over long periods of time at a predetermined level. Release periods may be as long as up to 90 days. The release of the active agent is controlled by adjusting the composition or properties of the hydrogel; i.e., by adjusting at least one of the anionic monomer content, crosslinker content, release media, and hydrogel surface area.

One factor that influences the release rate of the active agent is the amount of anionic monomer incorporated into the polymer hydrogel. FIG. 1 shows the release of cetylpyridinium chloride (CPC) into deionized water for polymer hydrogels having different amounts (0, 2.5, 5 and 10 mol %) of the anionic monomer AMPSA. A burst release of CPC is observed in the first 24 hours, with the exception of the hydrogel containing 10 mol % AMPSA (2 mol % DEGDMA crosslinker:10 mol % AMPSA:11.5 wt % CPC). Sample labels for the hydrogels in FIGS. 1-5 are expressed in terms of the ratio mol % DEGDMA:mol % AMPSA:wt % CPC. The initial burst seen in FIG. 1 is thought to arise from the release of CPC from the surface of the hydrogel. The CPC release is greatest for the polymer hydrogel containing no AMPSA. As the AMPSA concentration increases, the CPC release decreases. The suppressed release of CPC is largely due to the stoichiometry between the polyanion and the CPC cation. The molar ratio of AMPSA to CPC is 1:2.2 for the sample containing 2.5 mol % AMPSA and 11.5 wt % CPC. Increasing the AMPSA content to 5.0 mol % yields a 1 to 1 ratio between AMPSA and CPC. At 10 mol % AMPSA, the molar ratio between AMPSA and CPC is 1:0.5 and almost no CPC is released from the polymer matrix.

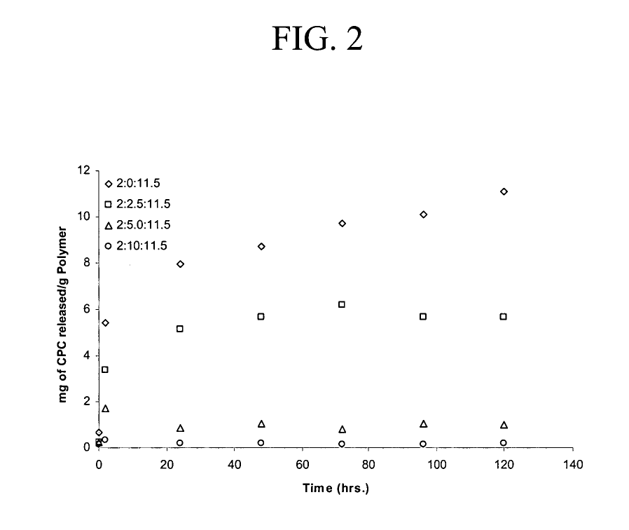

Another factor that influences the release of the active agent is the release media. FIG. 1 shows the release of CPC in deionized water, whereas FIG. 2 shows CPC release in pH 7.41 phosphate buffer. The same polymer formulations were used for both studies. The same release trends are observed in both studies: increasing the AMPSA concentration decreases the CPC release. However, the overall release of CPC is considerably suppressed in the phosphate buffer. After five days, the total release of CPC in phosphate buffer is only one-quarter of the total amount released in deionized water from the same polymer. Exchange between ions in solution and the polymeric complex—here, between cationic CPC and the polyanion—is established. This ion-exchange effect may suppress the release of the cationic material CPC in ionic solutions.

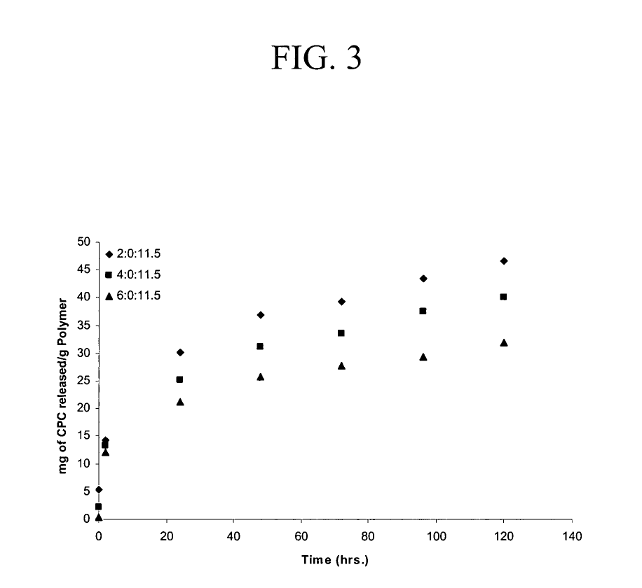

A third factor that influences the release of CPC is the amount of crosslinking in the hydrogel. Increasing the degree of crosslinking suppresses the release of the active agent from the polymer hydrogel. Compared to varying the anionic monomer content, varying the degree of crosslinking has a less dramatic effect on the release of the active agent and can be used to fine-tune release of the active agent from HEMA-based hydrogels. In the case of CPC, increasing the amount of DEGDMA crosslinker does not impact the CPC loading ability of these materials. In one example, a series of HEMA-based hydrogels, each having a CPC loading level of 11.5 wt %, were prepared with varying amounts (2, 4, and 6 mol %) of DEGDMA crosslinker. The release results, shown in FIG. 3, demonstrate that increasing the degree of crosslinking suppresses the release of CPC.

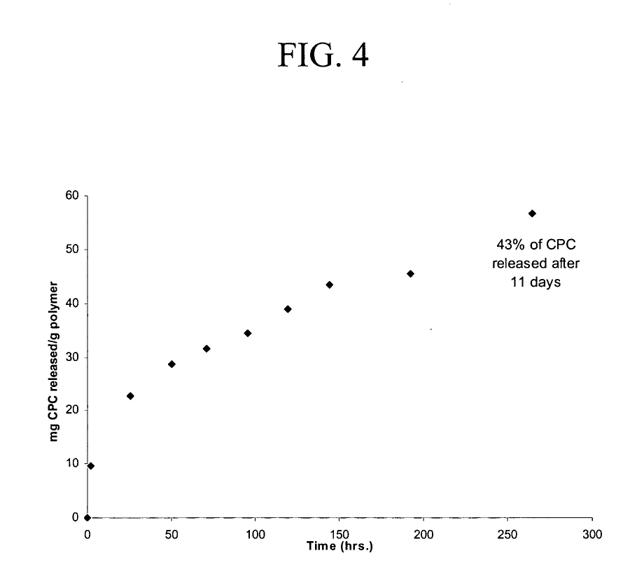

The dependence of long term release of CPC from a HEMA-based hydrogel with 2 mol % DEGDMA and 11.5 wt % CPC upon the amount of crosslinking present is shown in FIG. 4. Analysis of the solution after 15 days indicated a CPC concentration of 950 ppm, which corresponds the theoretical CPC concentration if all CPC incorporated into the hydrogel were released. The long-term release of CPC from a HEMA-based hydrogel with 4 mol % DEGDMA and 11.5 wt % CPC was also studied. After 11 days, 43% of the CPC was released. It was estimated that full release would be achieved after 35 days. The controlled and sustained release of CPC is clearly illustrated; by increasing the amount of crosslinker DEGDMA, the extended release time was more than doubled. Such controlled delivery of the active agent can be extended to up to 90 days.

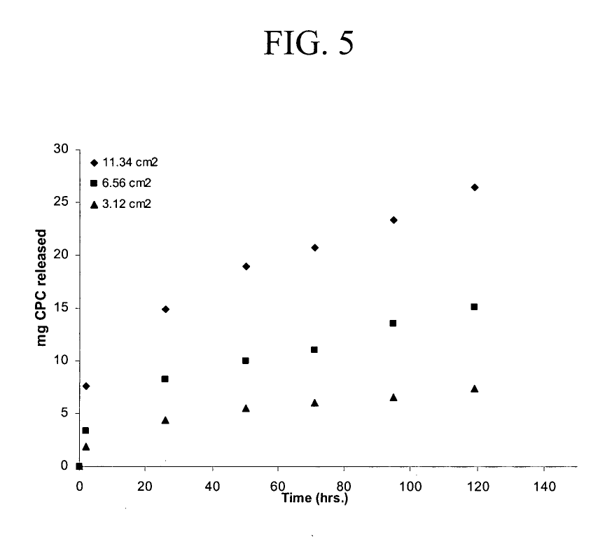

Hydrogel surface area also influences the release characteristics of the active agent. The release rate is proportional to the surface area of the material that is exposed to the release media or solution, with hydrogels having larger exposed surface areas having greater active agent release rates. The release of CPC is plotted from hydrogel disks having different surface areas is shown in FIG. 5. While the amount of CPC release per unit surface area is essentially the same for the samples measured, the amount of CPC released is dependent upon the exposed surface area.

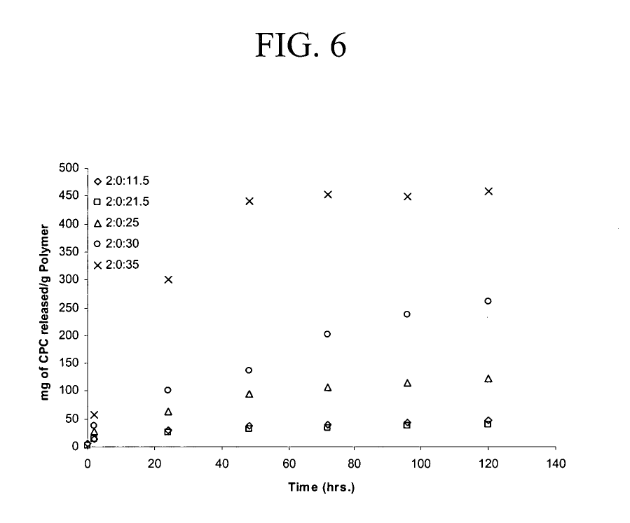

High levels of loading of the active agent may be achieved without compromising the physical integrity of the hydrogel. High levels of loading of the active agent enable quick delivery (i.e., within 24 hours or less) of the active agent or delivery that is sustainable over several days. Cetylpyridinium chloride loadings in HEMA-based hydrogel, for example, may be up to about 40 weight percent of the material. In one embodiment, CPC loadings range from about 11 wt % up to about 35 wt %. Higher CPC loadings are limited by the solubility of CPC. Data for the release of CPC are plotted as a function of CPC loading are shown in FIG. 6.

By employing photopolymerization techniques, discs or coatings of the material may be easily produced. The material may be shaped, molded, or formed into a film for an article comprising wood, plastic, metal or the like. In addition, the material may be used in dental resins or coatings, food processing and packaging, medical devices and implants, medical equipment, and water treatment applications.

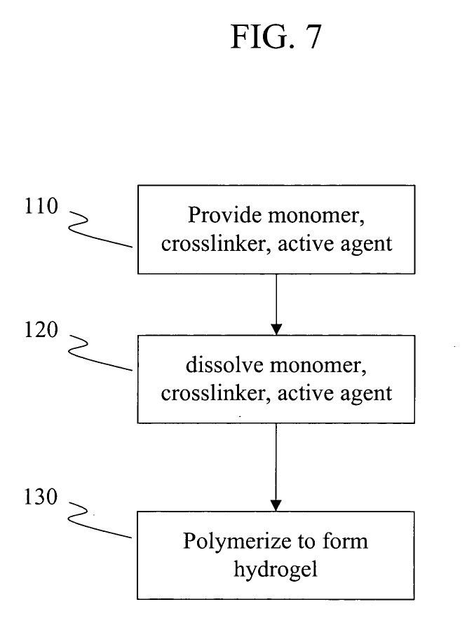

The invention also includes a method of making the material described herein. A flow chart illustrating the method is shown in FIG. 7. In method 100, a hydrogel is prepared by first providing an anionic monomer, a crosslinker, an initiator, and the active agent (Step 110). The active agent may comprise up to about 40 weight percent of the material. In one embodiment, loading levels of the active agent in the hydrogel range from about 11 wt % to about 40 wt %. In Step 120, the anionic monomer, crosslinker, initiator, and active agent are dissolved in water. In some instances, stirring or ultrasonic radiation may be used to facilitate dissolution. The solution may then be introduced into a mold or onto a surface of an article. In Step 130, polymerization is initiated, thus forming the hydrogel. Polymerization may be initiated, for example, by irradiating the solution with ultraviolet light.

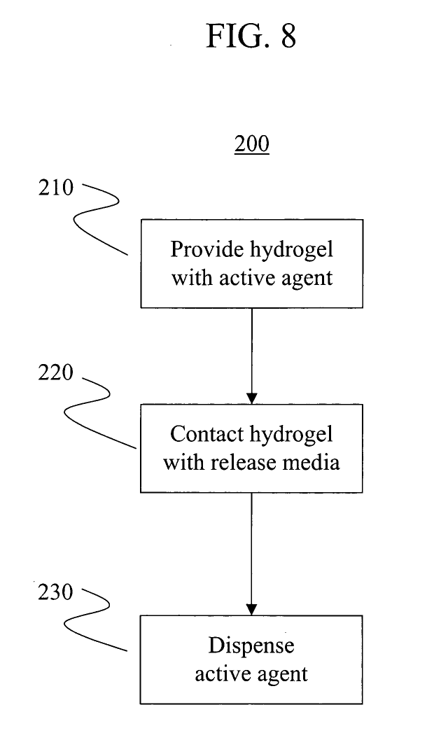

A method of dispensing an active agent is also provided by the invention. A flow chart of the method 200 is shown in FIG. 8. A hydrogel in which the active agent is dispersed is first provided in Step 210. The hydrogel containing the active agent is formed using the method described above and outlined in FIG. 7. The hydrogel containing the active agent is then brought into contact with a release medium (Step 220). The release medium is typically a liquid. Non-limiting examples of release media include, but are not limited to, deionized water, buffered solutions, or other aqueous media. Contact between the release medium and the hydrogel may be achieved by immersing the hydrogel in the medium, wetting a surface of the hydrogel, or by other means known in the art. Agitating the release medium and hydrogel may further facilitate release of the active agent. Once brought into contact with the release medium, the active agent is released from the hydrogel and dispensed (Step 230).

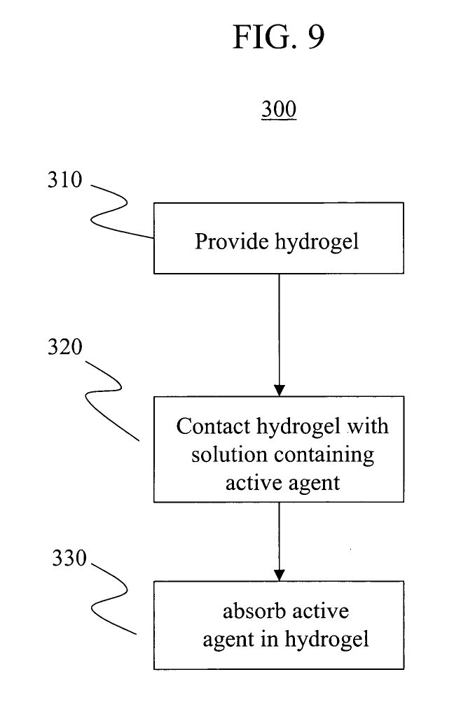

The invention also provides a method of absorbing an active agent from an aqueous solution. A flow chart illustrating the method 300 is shown in FIG. 9. In Step 310, a ‘blank’ hydrogel—i.e., a HEMA-based hydrogel that is substantially free of the active agent—is provided. The hydrogel is brought into contact with an aqueous solution containing the active agent (Step 320). Such contact is typically established by immersing the hydrogel in the solution. The rate and extent of absorption of the active agent in the hydrogel can be controlled by varying the amounts of monomer and crosslinker in the hydrogel. Once brought in contact with the aqueous solution, the active agent is absorbed by the hydrogel from the solution (Step 330). Absorption may occur under either static (i.e., without agitation of the solution) or dynamic conditions (i.e., with agitation of the solution)

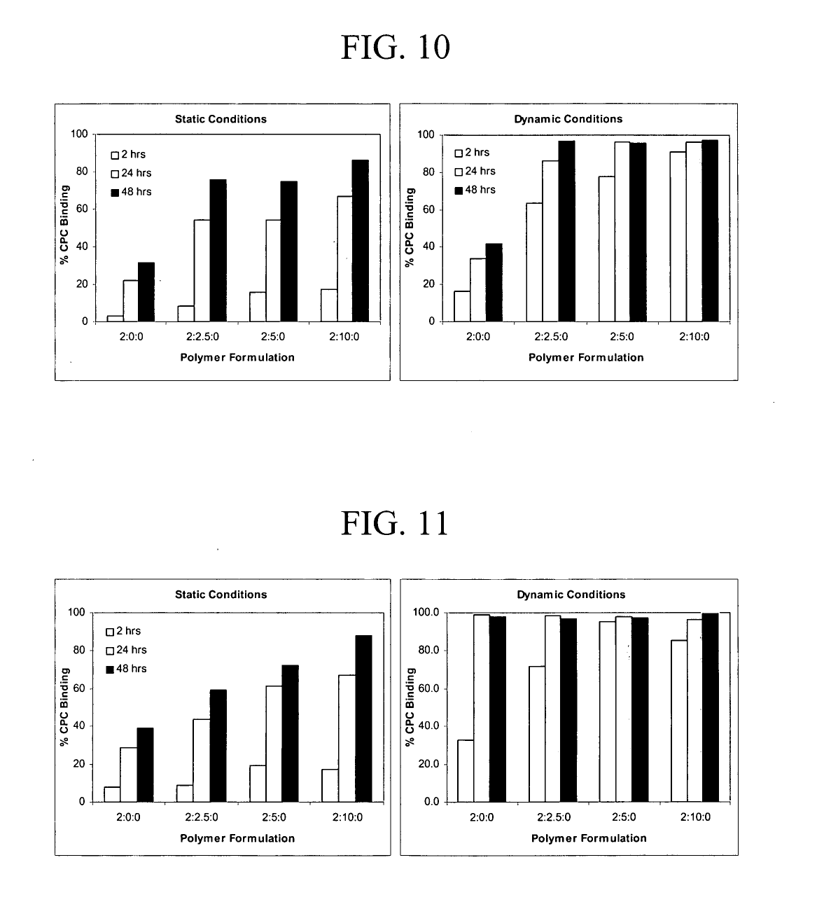

FIGS. 10 and 11 show the absorption of CPC by HEMA-based hydrogels, under static and dynamic conditions, from water and pH 7.41 phosphate buffer, respectively. The hydrogels each contain 2 mol % crosslinker DEGDMA and varying amounts of monomer AMPSA. The sample labels shown in FIGS. 8 and 9 represent the ratio mol % DEGDMA:mol % AMPSA:wt % CPC. Blank hydrogels with 2 mol % DEGDMA and varying degrees of AMPSA were prepared. The hydrogels were immersed in CPC solutions (in deionized water or pH 7.41 buffer) with an initial concentration of 75 ppm CPC. CPC binding was measured over 48 hours under both static and dynamic—i.e., with agitation—conditions. The most rapid absorption (t=2 hours) of CPC was observed under dynamic conditions for hydrogels containing AMPSA. After 2 hours, more than 60% of the CPC present was absorbed from water or buffer. Due to ionic interactions, CPC absorption increased as the amount of anionic monomer, AMPSA, increased in the hydrogel. The absorption results agree with release data where it was found that the stoichiometry between the polyanion and CPC cation largely controls release. In addition, CPC binding increased under dynamic conditions due to increased exposure of the hydrogel to the CPC solution. Absorption is also possible without the addition of AMPSA. This may be due to the hydrophobic nature of the CPC molecule and its affinity for the non-ionic hydrogel.

The following examples illustrate the features and advantages of the invention and are in no way intended to limit the invention thereto.

### EXAMPLE 1

### Preparation of Hydrogel Containing Cetylpyridinium Chloride (CPC)

Hydroxyethyl methacrylate (HEMA)-based hydrogels containing 2-acrylamido-2-methyl-1-propanesulfonic acid (AMPSA) were prepared with 0.3 mol % DMPA, either 2 or 4 mol % diethyleneglycol dimethyacrylate (DEGDMA), and 0, 2.5, 5, or 10 mol % 2,2-dimethoxy-2-phenylacetophenone (DMPA) to provide a total monomer (AMPSA), crosslinker (DEGDMA), and initiator (DMPA) content of 15 mmol. To fully dissolve the anionic monomer, 0.2 g of deionized water was added to the formulation. Based on the total mass of the monomer, crosslinker and initiator, approximately 11.5 wt % CPC was added. HEMA-based hydrogels without the anionic monomer AMPSA were also prepared with 0.3 mol % DMPA and 2, 4 or 6 mol % DEGDMA. CPC loading levels in these hydrogels ranged from 11.5 to 35 wt

In all cases, the polymer formulation was stirred or ultrasonically irradiated to dissolve all materials. The solution was pipetted into a circular Teflon® mold with a 0.050 in. (1.27 mm) depth and 0.500 in. (12.70 mm) diameter. Two additional Teflon® molds with a 0.050 in. (1.27 mm) depth were used to produce polymers with larger surface area. One mold had a diameter of 0.7500 in. (19.05 mm) and the other had a diameter of 1.0000 in. (25.40 mm).

Polymerization was initiated by irradiating the solutions in the molds for 5 minutes with a 4500 mW/cm² UV source in a black exposure box. Lamp intensity was monitored daily with a digital radiometer and sensor. The temperature within the exposure box was 40-45° C. The resulting hydrogel disks were hard and transparent. In addition, the hydrogels swelled uniformly in aqueous solutions and maintained their physical integrity even with high levels of CPC loading. The thickness of the disks was maintained at 0.050 in. (1.27 mm) to insure uniform photopolymerization across the thickness of the disk.

### EXAMPLE 2

### Burst Release

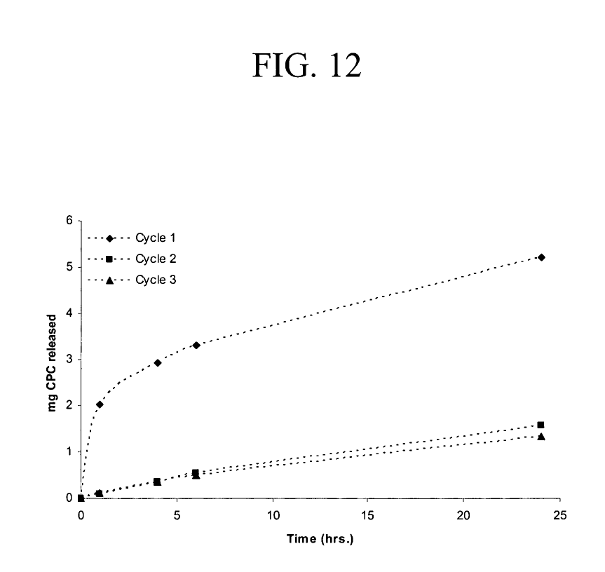

HEMA-based hydrogels comprising 4 mol % DEGDMA and 11.5 wt % CPC were prepared as described hereinabove. As shown in FIG. 12, an initial burst release was observed for the first release cycle. This initial burst may be attributed to CPC released from the surface of the hydrogel. After the first release cycle, the hydrogel was removed from solution and dried. The CPC release was reactivated in cycles 2 and 3 and followed a more stable, linear release profile. After the first cycle, approximately 22% of the total amount of CPC had been released. After the second cycle and third cycle, approximately 28% and 34% of the total amount of CPC had been released, respectively. After multiple release-drying cycles, the physical integrity of the hydrogel remained intact.

While typical embodiments have been set forth for the purpose of illustration, the foregoing description should not be deemed to be a limitation on the scope of the invention. Accordingly, various modifications, adaptations, and alternatives may occur to one skilled in the art without departing from the spirit and scope of the present invention.

1. A material for controlled release of an active agent, the material comprising:
a) a vehicle comprising a hydrogel, wherein the hydrogel comprises a hydrophobic portion and a hydrophilic portion and is substantially free of organic solvents; and
b) an active agent dispersed in the hydrogel, wherein the hydrogel releases the active agent in a controllable manner.

2. The material according to claim 1, wherein the hydrogel is one of a hydroxyethyl methacrylate-based hydrogel and a hydroxyl ethyl acrylate-based hydrogel.

3. The material according to claim 1, wherein the active agent is a biocide.

4. The material according to claim 3, wherein the biocide is one of an alkylpyridinium salt, an aliphatic quaternary ammonium salt, and a phenolic compound.

5. The material according to claim 4, wherein the alkylpyridinium salt comprises an alkyl group having from 8 to 18 carbon atoms and an anion capable of imparting water solubility.

6. The material according to claim 5, wherein the anion is one of a chloride anion, a bromide anion, and a sulfate anion.

7. The material according to claim 6, wherein the biocide is cetylpyridinium chloride.

8. The material according to claim 4, wherein the aliphatic quaternary ammonium salt has the structure 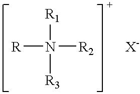
wherein R, independently and at each occurrence, is one of a benzyl group, a lower alkyl benzyl group, a C₁-C₄ alkyl group, and a C₁-C₄ hydroxyalkyl group; R₁, independently and at each occurrence, is one of a C₁-C₄ alkyl group and a C₁-C₄ hydroxyalkyl group; R₂, independently and at each occurrence, is a C₈-C₂₄ alkyl group; R₃, independently and at each occurrence, is one of a C₁-C₄ alkyl group, a C₁-C₄ hydroxyalkyl group, a C₈-C₂₄ alkyl group, and a C₈-C₂₄ hydroxyalkyl group, and wherein X⁻ is an anion capable of imparting water solubility to the aliphatic quaternary ammonium salt.

9. The material according to claim 8, wherein the aliphatic quaternary ammonium salt is one of (C₈-C₁₈) alkyl-dimethylbenzylammonium chloride and (C₈-C₁₈) alkyl-trimethylammonium bromide.

10. The material according to claim 4, wherein the phenolic compound is one of phenol, o-phenylphenol, 4-chloro-3,5-dimethylphenol, 2-chloro-3,5-dimethylphenol, m-cresol, o-cresol, p-cresol, and water-soluble salts thereof.

11. The material according to claim 3, wherein the biocide is one of benzalkonium (C₈-C₁₈) chloride, triclocarban, triclosan, chlorhexidene, chlorhexidene gluconate, hexachlorophene, phenolics, dibromopropamidine isethionate, 2-ethoxyethanol, 1-phenylethanol, 1,3,5-trioxane, formaldehyde, paraformaldehyde, o-phthaldehyde, and glutaraldehyde.

12. The material according to claim 1, wherein the hydrophobic portion comprises a compound having the formula CH₂CHR₁CO₂R₂ where R₁, independently and at each occurrence, is one of —H, —CH₃, and —CH₂CH₃, and R₂, independently and at each occurrence is one of an alkyl group, a cycloalkyl group, a hydroxyalkyl group, an aminoalkyl group, and an aryl group.

13. The material according to claim 1, wherein the hydrophobic portion is non-toxic.

14. The material according to claim 1, wherein the hydrophilic portion comprises a compound having a formula selected from the group consisting of:
a) CH₂CHR₁CONR₂R₃, where R₁, independently and at each occurrence, is one of —H, —CH₃, and —CH₂CH₃, R₂, independently and at each occurrence, is one of —H and an alkyl group, and R₃, independently and at each occurrence, is one of —H and an alkyl group;
b) CH₂CH₂PO₃R₂, where R₂, independently and at each occurrence, is one of —H, —CH₃ and —CH₂CH₃;
c) CH₂CH₂SO₃H; and
d) CH₂CHR₁CO₂R₂, where R₁, independently and at each occurrence, is one of —H, CH₃, and —CH₂CH₃, and R₂, independently and at each occurrence, is one of —H, —CH₃, and —CH₂CH₃.

15. The material according to claim 1, wherein the material is conformable to a substrate shape.

16. The material according to claim 1, wherein the material is moldable.

17. The material according to claim 1, wherein the material is capable of reactivation.

18. The material according to claim 1, wherein the material is capable of recharging with the active agent.

19. The material according to claim 1, wherein release of the portion of the active agent is controlled by the composition of the hydrogel.

20. The material according to claim 1, wherein the active agent is releasable over a predetermined time.

21. The material according to claim 21, wherein the predetermined time is up to 90 days.

22. The material according to claim 1, wherein the active agent is a cationic agent.

23. The material according to claim 22, wherein the cationic agent comprises at least one of a surfactant, a dye, and a therapeutic agent.

24. The material according to claim 23, wherein the surfactant is a biocide, wherein the biocide is one of an alkylpyridinium salt, an aliphatic quaternary ammonium salt, and a phenolic compound.

25. The material according to claim 23, wherein the dye is one of alizarin, alizarin red S monohydrate, bromocresol green, bromocresol purple, bromophenol blue, bromothymol blue, congo red, cresol red, methyl orange, and methyl red.

26. The material according to claim 23, wherein the therapeutic agent is one of an anti-inflammatory agent, a respiratory agent, an antibiotic, and cytarabine.

27. The material according to claim 1, wherein the material forms a film on an article.

28. The material according to claim 27, wherein the article comprises at least one of plastic, wood, and metal.

29. The material according to claim 1, wherein the active agent comprises up to about 40 weight percent of the material.

30. The material according to claim 29, wherein the active agent comprises between about 11 weight percent and about 40 weight percent of the material.

31. A vehicle for release of an active agent, the vehicle comprising a hydrogel selected from the group consisting of hydroxyethyl methacrylate-based hydrogels and hydroxyl ethyl acrylate-based hydrogels, wherein the hydrogel comprises a hydrophobic portion and a hydrophilic portion, and wherein the hydrogel regulates release of the active agent.

32. The vehicle according to claim 31, wherein the hydrophobic portion comprises a compound having the formula CH₂CHR₁CO₂R₂ where R₁, independently and at each occurrence, is one of —H, —CH₃, and —CH₂CH₃, and R₂, independently and at each occurrence is one of an alkyl group, a cycloalkyl group, a hydroxyalkyl group, an aminoalkyl group, and an aryl group.

33. The vehicle according to claim 31, wherein the hydrophobic portion is non-toxic.

34. The vehicle according to claim 31, wherein the hydrophilic portion comprises a compound having a formula selected from the group consisting of:
a) CH₂CHR₁CONR₂R₃, where R₁, independently and at each occurrence, is one of —H, —CH₃, and —CH₂CH₃, R₂, independently and at each occurrence, is one of —H and an alkyl group, and R₃, independently and at each occurrence, is one of —H and an alkyl group;
b) CH₂CH₂PO₃R₂, where R₂, independently and at each occurrence, is one of —H, —CH₃ and —CH₂CH₃;
c) CH₂CH₂SO₃H; and
d) CH₂CHR₁CO₂R₂, where R₁, independently and at each occurrence, is one of —H, CH₃, and —CH₂CH₃, and R₂, independently and at each occurrence, is one of —H, —CH₃, and —CH₂CH₃.

35. The vehicle according to claim 31, wherein the vehicle is rechargeable with the active agent.

36. The vehicle according to claim 31, wherein composition of the hydrogel regulates release of the active agent.

37. The vehicle according to claim 31, wherein vehicle releases the active agent over a predetermined time.

38. The vehicle according to claim 31, wherein the predetermined time is up to 90 days.

39. A material for controlled release of an active biocide, the material comprising:
a) a vehicle comprising a hydrogel selected from the group consisting of hydroxyethyl methacrylate-based hydrogels and hydroxyl ethyl acrylate-based hydrogels, wherein the hydrogel comprises a hydrophobic portion and a hydrophilic portion; and
b) an active biocide dispersed in the hydrogel, wherein the hydrogel regulates release of the active biocide.

40. A method of forming a material comprising a vehicle, the vehicle comprising a hydrogel selected from the group consisting of hydroxyethyl methacrylate-based hydrogels and hydroxyl ethyl acrylate-based hydrogels, and an active agent dispersed in the hydrogel, the method comprising the steps of:
a) providing a monomer, a crosslinker, an initiator, and the active agent;
b) forming an aqueous solution of the monomer, the crosslinker, the initiator, and the active agent; and
c) forming the hydrogel by polymerizing the monomer in the solution, wherein the active agent is dispersed throughout the hydrogel.

41. A method of dispensing an active agent, the method comprising the steps of:
a) providing a vehicle, the vehicle comprising a hydrogel selected from the group consisting of hydroxyethyl methacrylate-based hydrogels and hydroxyl ethyl acrylate-based hydrogels and the active agent dispersed in the hydrogel; and
b) contacting the vehicle with an aqueous release medium, wherein the active agent is released from the vehicle into the release medium.

42. A method of absorbing an active agent from an aqueous medium containing the active agent, the method comprising the steps of:
a) providing a hydrogel selected from the group consisting of hydroxyethyl methacrylate-based hydrogels and hydroxyl ethyl acrylate-based hydrogels; and
b) contacting the hydrogel with the aqueous medium, wherein the active agent is absorbed by the hydrogel from the aqueous medium.
